(19) United States
(12) Patent Application Publication
(10) Pub. No.: US 2015/0083924 A1
(43) Pub. Date: Mar. 26, 2015

(54) RADIOGRAPHIC IMAGING DEVICE AND RADIATION DETECTOR

(71) Applicant: FUJIFILM CORPORATION, Tokyo (JP)

(72) Inventors: Yoshihiro OKADA, Kanagawa (JP); Takaaki ITO, Kanagawa (JP)

(21) Appl. No.: 14/555,604

(22) Filed: Nov. 27, 2014

Related U.S. Application Data
(63) Continuation of application No. PCT/JP2013/064673, filed on May 27, 2013.

(30) Foreign Application Priority Data
May 30, 2012 (JP) 2012-123627

(51) Int. Cl. H01L 27/146; G01T 1/17; G01T 1/24

(52) U.S. Cl. CPC H01L 27/14659; G01T 1/247; G01T 1/17; H01L 27/14663
USPC 250/370.08; 250/370.14

(57) ABSTRACT

First TFTs are provided in correspondence with respective intersection portions between plural signal lines and plural first scan lines. Control terminals of the first TFTs are connected to the corresponding first scan lines, and output terminals of the first TFTs are connected to the corresponding signal lines. Sensors are connected to input terminals of the first TFTs. Second TFTs include input terminals that are connected to respective sensors and control terminals that are connected to second scan lines. Output terminals of second TFTs whose input terminals are connected to a plural number of the sensors, which sensors are adjacent in a first direction and a second direction, are connected to the same signal line. A plural number of the second scan lines that are provided with driving signals that are identical or the same are electrically connected to one another by a redundant line.

### CROSS-REFERENCE TO RELATED APPLICATIONS

This application is a continuation application of International Application No. PCT/JP2013/064673, filed May 27, 2013, the disclosure of which is incorporated herein by reference in its entirety. Further, this application claims priority from Japanese Patent Application No. 2012-123627, filed May 30, 2012, the disclosure of which is incorporated herein by reference in its entirety.

### BACKGROUND OF THE INVENTION

1. Field of the Invention

The present invention relates to a radiographic imaging device and radiation detector that generate a radiation image based on radiation passing through an imaging target region.

2. Description of the Related Art

In recent years, radiation detectors such as flat panel detectors (FPD) and the like have been realized. In an FPD, a radiation-sensitive layer is disposed on a thin film transistor (TFT) active matrix substrate. The FPD converts radiation directly to digital data. A portable radiographic imaging device (hereinafter referred to as an “electronic cassette”) that incorporates a radiation detector, electronic circuits including an image memory, and a power supply section, and that stores radiation image data from the radiation detector in the image memory, has also been realized. There are calls for the same radiation detector to be used for imaging both still images and video images (fluoroscopic images). In general, in cases of imaging still images, there are many cases in which a high definition image (high resolution) is required but a low frame rate (imaging interval) is acceptable. On the other hand, in cases of video imaging, there are many cases in which a high frame rate is required but a low resolution is acceptable.

Accordingly, there are technologies that enable the acquisition of high frame rate images or the acquisition of high definition images in accordance with objectives, such as, for example, the technology recited in Japanese Patent Application Laid-Open (JP-A) No. 2004-46143. JP-A No. 2004-46143 recites an image forming device that is provided with pixels arrayed in a two-dimensional matrix, a signal processing circuit part 15 that processes signals from the pixels, and a gate driver circuit part 17 that controls connections with the pixels. The gate driver circuit part 17 is connected to the pixels by gate lines 13A and 13B. The gate lines 13A and 13B include gate lines that are respectively connected to each pixel in a row or a column and gate lines that connect to pixels in plural rows or plural columns in common.

According to the technology recited in JP-A No. 2004-46143, if a switching element connected to a gate line of system A, which connects to all the pixels belonging to the same row, is driven, an image with a usual number of pixels is outputted, whereas if a switching element connected to a gate line of system B, which connects to all pixels in common across a plural number of rows, is driven, an image with one pixel for four of the usual pixels is outputted.

Thus, in a case in which four pixels at a time of high definition imaging become one pixel at a time of high speed driving, a defect that would correspond to one pixel has a size of four pixels. Therefore, in order to maintain consistent image quality at times of high-speed driving, a standard for determining whether or not a defect is acceptable must be specified more strictly, and it is difficult to maintain productivity. In particular, in a case in which there is a breakage in a gate line (a scan line), all pixels from the breakage portion of the gate line to an end portion become defective pixels, and the size of the defect is remarkably large.

The present invention provides a radiographic imaging device and a radiation detector in which a resolution may be switched and that may prevent the occurrence of defective pixels in a case in which a breakage occurs in a scan line.

### SUMMARY OF THE INVENTION

A first aspect of the present invention is a radiographic imaging device including: a plurality of first scan lines and a plurality of second scan lines extending in a first direction; a plurality of signal lines extending in a second direction that crosses the first direction; a plurality of first switching elements provided in correspondence with intersection portions between the plurality of signal lines and the plurality of first scan lines, control terminals of the first switching elements being connected to the corresponding first scan lines and output terminals of the first switching elements being connected to the corresponding signal lines; a plurality of sensors, each of which is connected to an input terminal of a respective one of the first switching elements and that generates charges in accordance with intensities of irradiated radiation or intensities of light corresponding to the radiation; a plurality of second switching elements, each of which includes an input terminal connected to a respective one of the sensors and a control terminal connected to one of the second scan lines, respective output terminals of the plurality of the second switching elements, whose respective input terminals are connected to a plurality of the sensors, which sensors are adjacent in the first direction and the second direction, being connected to the same signal line; a first driving signal provision section that provides sequential driving signals to the plurality of first scan lines; a second driving signal provision section that provides sequential driving signals to the plurality of second scan lines; and a connection portion that electrically connects to one another a plurality of the second scan lines to which the same or identical driving signals are provided by the second driving signal provision section.

In a second aspect of the present invention, in the first aspect, the connection portion may be provided at second end portions of the second scan lines, the second end portions being at an opposite side of the second scan lines from first end portions at a side thereof at which the second driving signal provision section is connected. In a third aspect of the present invention, in the second aspect, the connection portion may be provided at the first end portions and the second end portions of the second scan lines. In a fourth aspect of the present invention, in the second or third that aspect, the connection portion may be provided between the first end portions and the second end portions of the second scan lines.

In a fifth aspect of the present invention, in the first aspect, the connection portion may be formed integrally with each of the second scan lines. In a sixth aspect of the present invention, in the first to fourth aspects, the connection portion may include a flexible member.

In a seventh aspect of the present invention, in the above aspects, the first driving signal provision section may provide driving signals to each of the first scan lines when in a first imaging mode, and the second driving signal provision section may provide driving signals to each of the second scan lines when in a second imaging mode.

In an eighth aspect of the present invention, in the above aspects, the first driving signal provision section and the second driving signal provision section may be formed in a single package. In a ninth aspect of the present invention, in the eighth aspect, the first driving signal provision section and the second driving signal provision section may be connected to end portions of the plurality of first scan lines and the plurality of second scan lines.

In a tenth aspect of the present invention, in the first to seventh aspects, the first driving signal provision section and the second driving signal provision section may be separately provided. In an eleventh aspect of the present invention, in the tenth aspect, the first driving signal provision section may be connected to the plurality of first scan lines at end portions at the opposite side thereof from connection portions that connect the plurality of second scan lines with the second driving signal provision section.

A twelfth aspect of the present invention, in the above aspects, may further include a signal processing section that is connected to each of the plurality of signal lines and that generates a radiation image in accordance with charges read out to the signal lines from the plurality of sensors in response to driving that turns on the first switching elements or the second switching elements.

A thirteenth aspect of the present invention is a radiation detector including: a plurality of first scan lines and a plurality of second scan lines extending in a first direction; a plurality of signal lines extending in a second direction that crosses the first direction; a plurality of first switching elements provided in correspondence with intersection portions between the plurality of signal lines and the plurality of first scan lines, control terminals of the first switching elements being connected to the corresponding first scan lines and output terminals of the first switching elements being connected to the corresponding signal lines; a plurality of sensors, each of which is connected to an input terminal of a respective one of the first switching elements and that generates charges in accordance with intensities of irradiated radiation or intensities of light corresponding to the radiation; a plurality of second switching elements, each of which includes an input terminal connected to a respective one of the sensors and a control terminal connected to one of the second scan lines, respective output terminals of the plurality of the second switching elements, whose respective input terminals are connected to a plurality of the sensors, which sensors are adjacent in the first direction and the second direction, being connected to the same signal line; and a connection portion that electrically connects to one another a plurality of the second scan lines to which the same or identical driving signals are provided.

According to the radiographic imaging device and radiation detector relating to the present invention, an occurrence of defective pixels may be prevented even in a case in which a breakage in a scan line occurs.

### BRIEF DESCRIPTION OF DRAWINGS

Detailed explanation follows regarding exemplary embodiments of the present invention, with reference to the following drawings.

FIG. 1 is a block diagram showing the configuration of a radiographic imaging system in accordance with an exemplary embodiment of the present invention.

FIG. 2 is a perspective view showing configurations of an electronic cassette that is one aspect of a radiographic imaging device in accordance with the exemplary embodiment of the present invention.

FIG. 3 is a sectional view showing the configurations of the electronic cassette that is one aspect of the radiographic imaging device in accordance with the exemplary embodiment of the present invention.

FIG. 4 is a sectional view for explaining penetration side sampling and irradiation side sampling.

FIG. 5 is a diagram showing electronic configurations of the radiographic imaging device in accordance with the exemplary embodiment of the present invention.

FIG. 6 is a diagram showing connection configurations between a radiation detector and a scan line driving circuit in accordance with the exemplary embodiment of the present invention.

FIG. 7 is a timing chart of driving signals in a high resolution mode of the radiographic imaging device in accordance with the exemplary embodiment of the present invention.

FIG. 8 is a timing chart of driving signals in a low resolution mode of the radiographic imaging device in accordance with the exemplary embodiment of the present invention.

FIG. 9 is a partial structural diagram of the radiation detector illustrating a case in which a breakage in a second scan line has occurred.

FIG. 10 is a diagram showing electronic configurations of a radiographic imaging device in accordance with an exemplary embodiment of the present invention.

FIG. 11 is a diagram showing connection configurations between a radiation detector and a scan line driving circuit in accordance with the exemplary embodiment of the present invention.

FIG. 12 is a diagram showing electronic configurations of a radiographic imaging device in accordance with an exemplary embodiment of the present invention.

FIG. 13 is a diagram showing electronic configurations of a radiographic imaging device in accordance with an exemplary embodiment of the present invention.

FIG. 14 is a diagram showing electronic configurations of a radiographic imaging device in accordance with an exemplary embodiment of the present invention.

### DESCRIPTION OF EMBODIMENTS

Herebelow, exemplary embodiments of the present invention are described in detail while referring to the drawings. Elements or portions that are substantially the same or equivalent are assigned the same reference numerals in the respective drawings.

### First Exemplary Embodiment

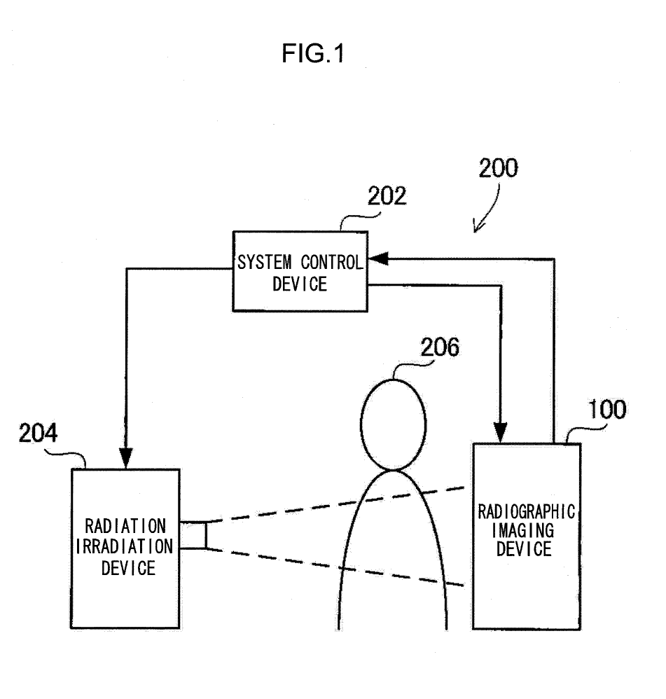

FIG. 1 is a block diagram showing the configuration of a radiographic imaging system in accordance with an exemplary embodiment of the present invention.

A radiographic imaging system 200 includes a radiographic imaging device 100, a radiation irradiation device 204 and a system control device 202. The radiation irradiation device 204 irradiates radiation (for example, X-rays or the like) at an imaging subject 206. The radiographic imaging device 100 generates radiation images that visualize radiation that has been irradiated from the radiation irradiation device 204 and passed through an imaging subject 206. The system control device 202 commands the radiographic imaging device 100 and the radiation irradiation device 204 to image radiation images, and acquires radiation images generated by the radiographic imaging device 100. The radiation irradiation device 204 irradiates radiation in accordance with control signals provided from the system control device 202. Radiation that has passed through the imaging subject 206, which is disposed at an imaging position, is irradiated onto the radiographic imaging device 100.

The radiographic imaging device 100 images radiation images in different imaging modes: a high resolution mode and a low resolution mode. The high resolution mode is a mode that images radiation images with high resolution, and is applicable to, for example, imaging still images. The low resolution mode is a mode that images radiation images at a high frame rate but with a lower resolution than images generated in the high resolution mode, and is applicable to, for example, imaging video images. The system control device 202 provides commands to the radiographic imaging device 100 instructing which of the high resolution mode and the low resolution mode to select in accordance with, for example, instructions from a user.

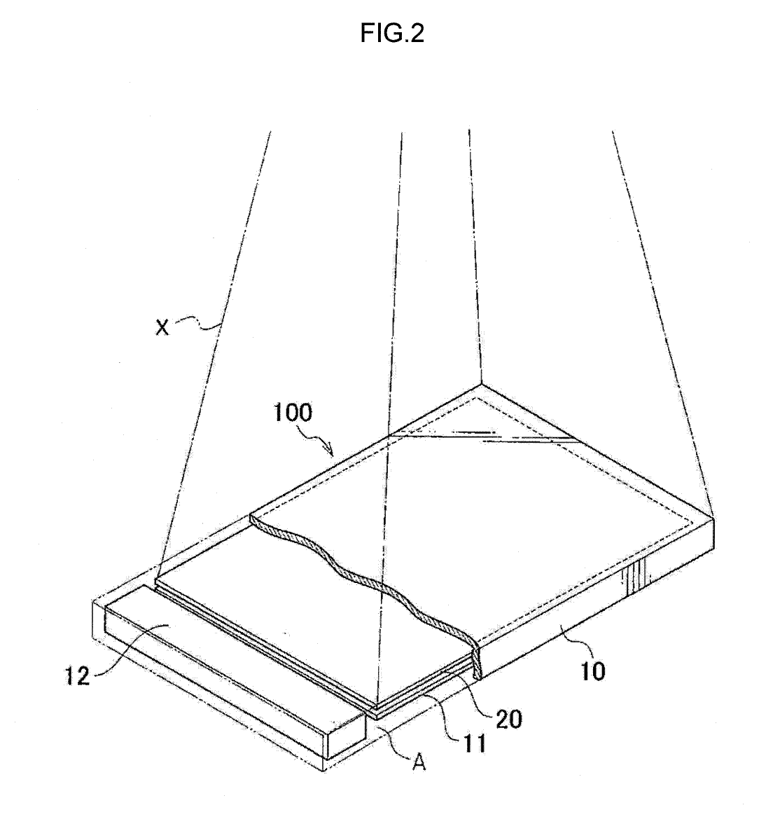

FIG. 2 is a perspective view showing configurations of the radiographic imaging device 100 in accordance with the present exemplary embodiment. The radiographic imaging device 100 according to the present exemplary embodiment takes the form of an electronic cassette. The radiographic imaging device 100 is provided with a casing 10 formed of a material that transmits the radiation, and the radiographic imaging device 100 is configured to be waterproof and tightly sealed. A space A that accommodates various components is formed inside the casing 10. Inside the space A, a radiation detector 20 that detects radiation X passing through the imaging subject and a lead plate 11 that absorbs back scattering of the radiation X are arranged in this order from an irradiated surface side of the casing 10 on which the radiation X is irradiated. A case 12 is disposed at one end of the interior of the casing 10. The case 12 accommodates a power supply section and the like (not shown in the drawings) at a location that does not overlap with the radiation detector 20.

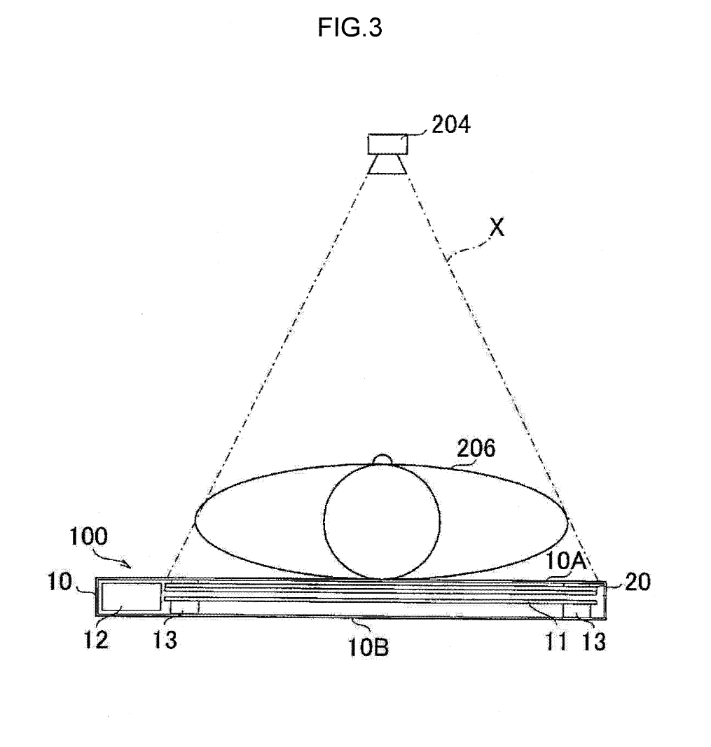

As shown in FIG. 3, a support body 13 is disposed inside the casing 10 at the inner face of a rear face portion 10B, which opposes a top plate 10A. Between the support body 13 and the top plate 10A, the radiation detector 20 and the lead plate 11 are arranged in this order in the direction of irradiation of the radiation X. With a view to weight reduction and tolerating dimensional variations, the support body 13 is configured of, for example, a foam material. The support body 13 supports the lead plate 11.

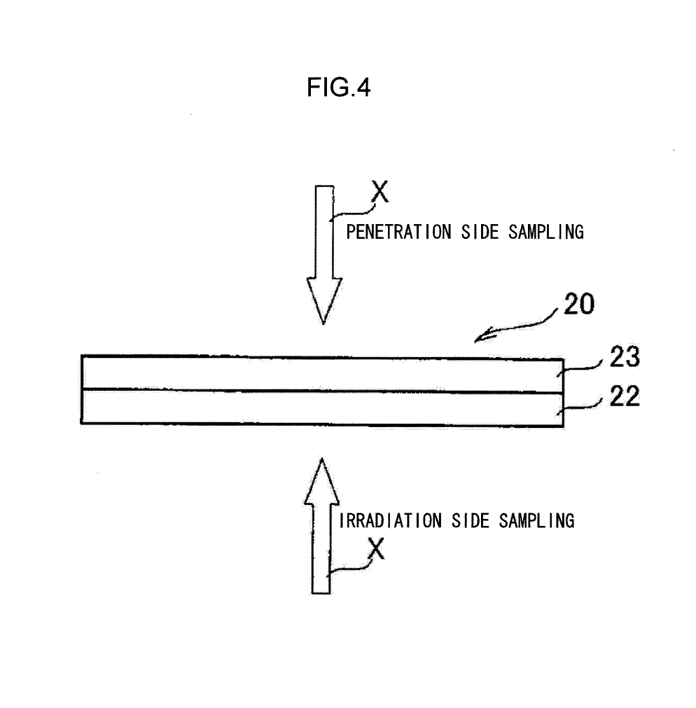

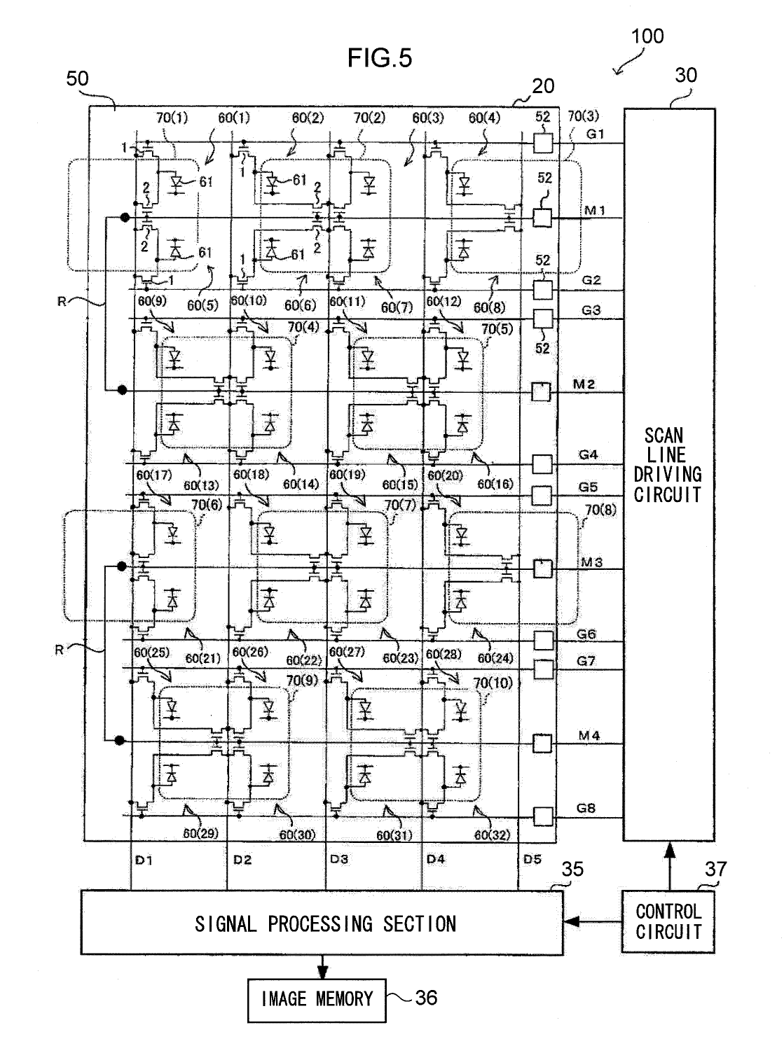

FIG. 4 is a sectional diagram schematically showing a layer configuration of the radiation detector 20 in accordance with the present exemplary embodiment. The radiation detector 20 has a configuration in which a TFT substrate 22 and a scintillator 23 are layered. The TFT substrate 22 includes, on a glass substrate 50, sensors 61, which are described below, thin film transistors (TFTs 1 and TFTs 2) and the like (see FIG. 5). The scintillator 23 includes a fluorescent material that converts irradiated radiation to light and emits the light.

In a case in which, as shown in FIG. 4, the radiation is irradiated from the side of the radiation detector 20 at which the scintillator 23 is formed and the radiation detector 20 reads the radiation image with the TFT substrate 22, which is referred to as penetration side sampling (PSS), light is more strongly emitted from the side of the scintillator 23 of the face thereof on which the radiation is irradiated. In a case in which the radiation is irradiated from the side of the radiation detector 20 at which the TFT substrate 22 is formed and the radiation detector 20 reads the radiation image with the TFT substrate 22 provided at the side of the front face that is the face on which the radiation is incident, which is referred to as irradiation side sampling (ISS), light is more strongly emitted from the side of the scintillator 23 of the face thereof that is joined to the TFT substrate 22. The below-described sensors 61 provided at the TFT substrate 22 receive the light produced by the scintillator 23 and generate electronic charges. Therefore, in a case in which the radiation detector 20 is of an ISS type, light emission positions of the scintillator 23 are closer to the TFT substrate 22 than in a case in which the radiation detector 20 is of a PSS type. As a result, the resolution of the radiation images obtained by imaging is higher.

FIG. 5 is a structural diagram showing electronic configurations of the radiographic imaging device 100 in accordance with the present exemplary embodiment. As shown in FIG. 5, the radiographic imaging device 100 includes the radiation detector 20, a scan line driving circuit 30, a signal processing circuit 35, an image memory 36 and a control circuit 37. Note that, in FIG. 5, the scintillator 23 is not shown in the drawing.

The radiation detector 20 includes a plural number of pixels 60 two-dimensionally arrayed on a glass substrate 50 in a predetermined first direction and a second direction that crosses the first direction. Each of the plural pixels 60 includes one of the sensors 61, a first thin film transistor 1 (hereinafter referred to as the TFT 1), and a second thin film transistor 2 (hereinafter referred to as the TFT 2). The sensor 61 is formed with a photoelectric conversion element that receives light emitted from the scintillator 23 in response to the irradiation of radiation and generates charges, and that accumulates the generated charges. The first thin film transistor 1 (hereinafter referred to as the TFT 1) and the second thin film transistor 2 (hereinafter referred to as the TFT 2) read the charges accumulated in the sensor 61 out to a signal line D.

In each pixel 60, the input terminals of the TFT 1 and the TFT 2 are connected to the sensor 61. The TFTs 1 are switching elements that are driven when a radiation image is being imaged in the high resolution mode, and the TFTs 2 are switching elements that are driven when a radiation image is being imaged in the low resolution mode. In FIG. 5, the arrangement of the pixels 60 is shown simplified; the pixels 60 are arranged in lines of, for example, 1024 in the first direction and in the second direction (that is, 1024 by 1024 pixels). The sensors 61 of the pixels 60 are connected to common lines, which are not shown in the drawings, forming a configuration such that a bias voltage is applied via the common lines from a power supply section (not shown in the drawings).

The TFT substrate 22 includes plural first scan lines G (illustrated by lines G1 to G8 in FIG. 5), plural second scan lines M (illustrated by lines M1 to M4 in FIG. 5), and plural signal lines D (illustrated by lines D1 to D5 in FIG. 5) on the glass substrate 50. The first scan lines G and the second scan lines M extend in the first direction along the array of the pixels 60. The signal lines D extend in the second direction orthogonally to the scan lines G and M. The scan lines G and the signal lines D are provided in correspondence with rows and columns of the pixels 60. For example, in the case of an array of 1024 by 1024 of the pixels 60, 1024 each of the first scan lines G and the signal lines D are provided. In the present exemplary embodiment, the second scan lines M are half the number of the first scan lines G. That is, in the aforementioned case, 512 of the second scan lines M are provided.

The control terminals (gates) of the plural TFTs 1 that are driven when a radiation image is being imaged in the high resolution mode are connected to the respective first scan lines G. More specifically, the control terminals (gates) of the TFTs 1 in plural pixels 60 that are in a line along the direction in which the first scan lines G extend are connected to the same first scan line G. For example, in the example shown in FIG. 5, the control terminals (gates) of the TFTs 1 in pixels 60(1) to 60(4) are connected to the first scan line G1, and the control terminals (gates) of the TFTs 1 in pixels 60(5) to 60(8) are connected to the first scan line G2.

The control terminals (gates) of a plural number of the TFTs 2 that are driven when a radiation image is being imaged in the low resolution mode are connected to each of the second scan lines M. More specifically, the control terminals (gates) of the TFTs 2 in plural pixels 60 that are in a line along the direction in which the second scan lines M extend and the TFTs 2 in pixels 60 that are adjacent thereto in the direction of extension of the signal lines D, are all connected to the same second scan line M. For example, in the example shown in FIG. 5, the control terminals (gates) of the TFTs 2 in pixels 60(1) to 60(8) are connected to the second scan line M1, and the control terminals (gates) of the TFTs 2 in pixels 60(9) to 60(16) are connected to the second scan line M2.

The output terminals of the TFTs 1 in a plural number of the pixels 60 that are in a line along the direction in which the signal lines D extend are connected to the same signal line D. For example, in the example shown in FIG. 5, the output terminals of the TFTs 1 in pixels 60(1), 60(5), 60(9), 60(13), 60(17), 60(21), 60(25) and 60(29) are connected to the signal line D1, and the output terminals of the TFTs 1 in pixels 60(2), 60(6), 60(10), 60(14), 60(18), 60(22), 60(26) and 60(30) are connected to the signal line D2.

The output terminals of the TFTs 2 in four pixels that are adjacent to one another in the direction in which the scan lines G and M extend and the direction in which the signal lines D extend, and that are connected to the same second scan line M, are connected to the same signal line D. For example, in the example shown in FIG. 5, the output terminals of the TFTs 2 that constitute a composite pixel 70(4) formed of the pixels 60(9), 60(10), 60(13) and 60(14) are connected to the signal line D2, and the output terminals of the TFTs 2 that constitute a composite pixel 70(2) formed of the pixels 60(2), 60(3), 60(6) and 60(7) are connected to the signal line D3.

The scan line driving circuit 30 is provided at one of two adjacent edges of the radiation detector 20. The signal processing section 35 is provided at the other of the two adjacent edges. Each of the first scan lines G and each of the second scan lines M is connected to the scan line driving circuit 30 via a respective connection terminal 52.

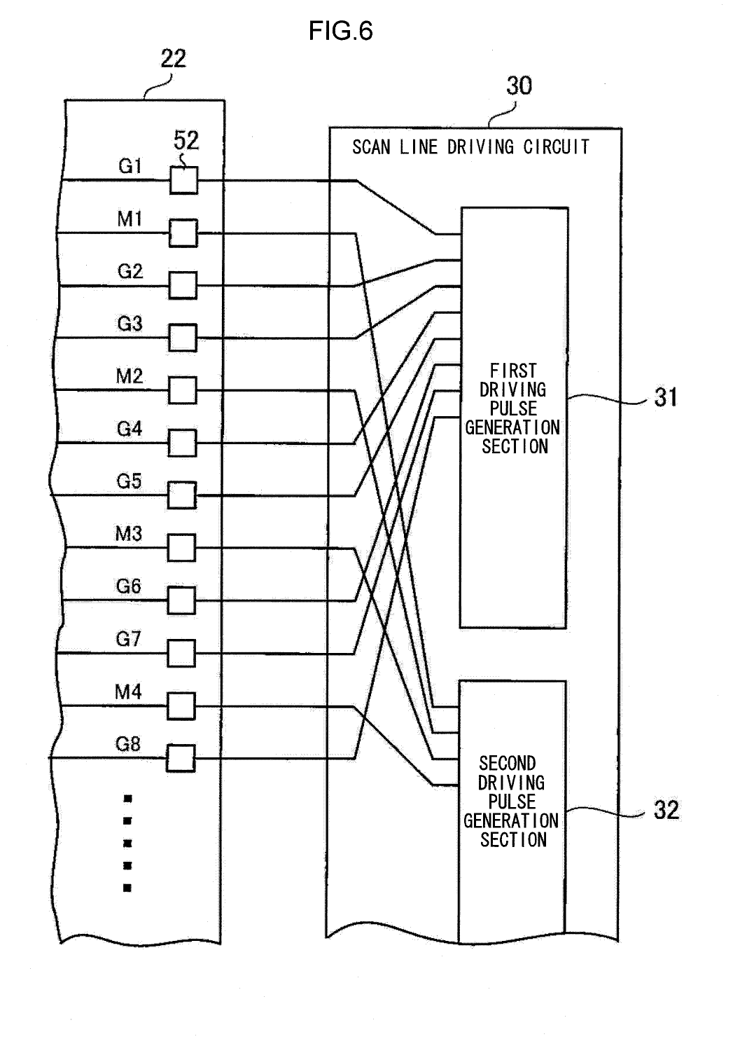

FIG. 6 is a diagram showing connection configurations between the radiation detector 20 and the scan line driving circuit 30. The scan line driving circuit 30 includes a first driving signal generation circuit 31, which generates driving signals when in the high resolution mode, and a second driving signal generation circuit 32, which generates driving signals when in the low resolution mode. The first driving signal generation circuit 31 and the second driving signal generation circuit 32 are accommodated in a single integrated circuit or a single semiconductor package and are formed integrally.

The first driving signal generation circuit 31 includes a shift register circuit. The first driving signal generation circuit 31 is connected to each of the first scan lines G via the respective connection terminals 52, and sequentially outputs driving pulses to each of the first scan lines G when in the high resolution mode. The TFTs 1 turn ON in response to the driving pulses provided via the first scan lines G, and output charges accumulated in the sensors 61 to the signal lines D.

The second driving signal generation circuit 32 also includes a shift register circuit. The second driving signal generation circuit 32 is connected to each of the second scan lines M via the respective connection terminals 52, and sequentially outputs driving pulses to each of the second scan lines M when in the low resolution mode. The TFTs 2 turn ON in response to the driving pulses provided via the second scan lines M, and output charges accumulated in the sensors 61 to the signal lines D.

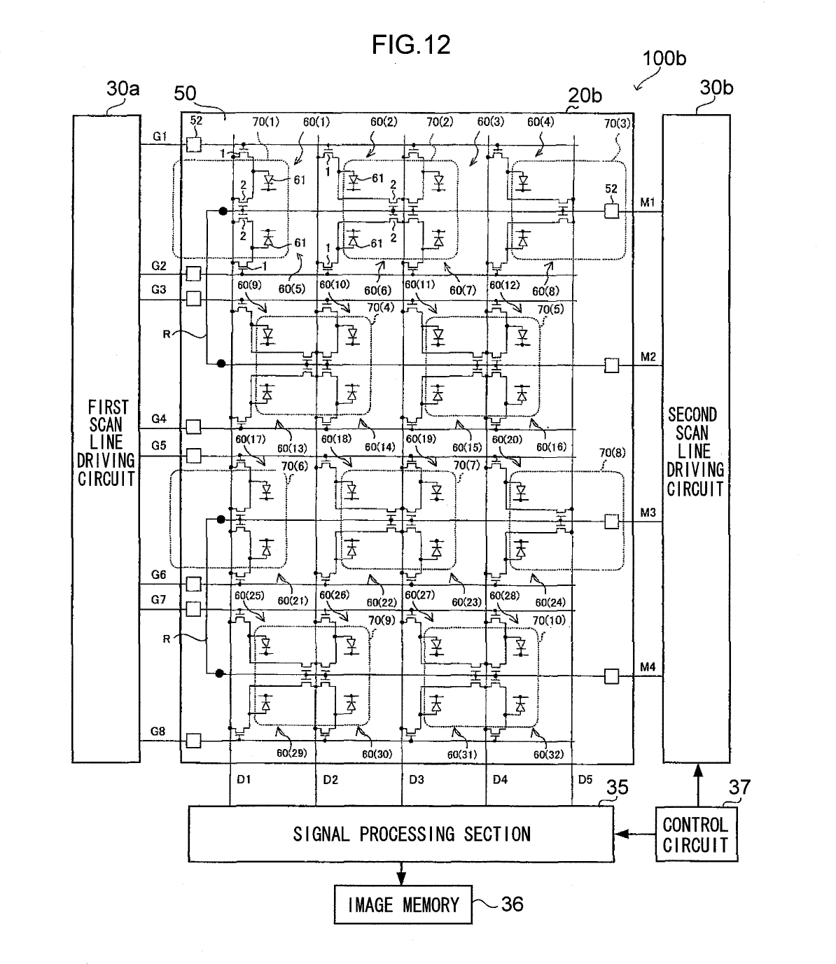

Thus, in the present exemplary embodiment, the first driving signal generation circuit 31 that operates in the high resolution mode and the second driving signal generation circuit 32 that operates in the low resolution mode are accommodated in the single scan line driving circuit 30. Because the scan line driving circuit 30 is a single configuration, an imaging area may be enlarged compared to a case in which a scan line driving circuit is provided as plural configurations at both sides of the radiation detector 20 (see FIG. 12), or the overall size of the radiographic imaging device 100 may be reduced. The first driving signal generation circuit 31 and the second driving signal generation circuit 32 may also be separated and disposed at one side of the radiation detector 20. In this case, some ingenuity is required for routing the first scan lines G and the second scan lines M on the glass substrate 50, as a result of which the wiring burden is greater and artifacts may result. In a case in which the radiographic imaging device 100 is employed as a portable electronic cassette, as in the present exemplary embodiment, it is desirable to form the scan line driving circuit 30 as a single configuration that is capable of handling both the high resolution mode and the low resolution mode, and providing the scan line driving circuit 30 at only one side of the radiation detector 20 is desirable with regard to assuring the imaging area and reducing the size, and in regard to avoiding an increase in the wiring burden.

In FIG. 5, a configuration is illustrated in which the single scan line driving circuit 30 is provided for all of the scan lines G and M. However, scan line driving circuits may be respectively provided for sets of predetermined numbers of scan lines. For example, in the case in which 1024 of the first scan lines G are provided on the glass substrate 50, scan line driving circuits may be provided for sets of 256 thereof, in which case four of the scan line driving circuits are provided. The same possibility applies to the signal processing section 35.

In the present exemplary embodiment, as described below, driving pulses with the same time width and the same signal level are provided from the scan line driving circuit 30 to the second scan lines M1 and M2 simultaneously. The second scan lines M1 and M2 that constitute a pair to which the same driving signals are provided simultaneously are electrically connected to one another by a redundant line R, at end portions of the scan lines M1 and M2 that are at the opposite side thereof from end portions at the side at which the scan line driving circuit 30 is disposed. Similarly, the second scan lines M3 and M4 constitute a pair to which driving signals with the same time width and the same signal level are simultaneously provided from the scan line driving circuit 30. The pair formed of the second scan lines M3 and M4 are electrically connected to one another by a redundant line R at end portions of the scan lines M3 and M4 that are at the opposite side thereof from end portions at the side at which the scan line driving circuit 30 is disposed. The redundant lines R are functionally unnecessary for the radiation detector 20 to image radiation images. However, as described below, the redundant lines R prevent the occurrence of defective pixels when there is a breakage in a second scan line M. Herein, the first and second scan lines G and M, the signal lines D and the redundant lines R may be formed by, for example, using vapor deposition, sputtering or the like to form a film of a conductive material such as aluminium or the like on the glass substrate 50, and then patterning the film. In this case, the redundant lines R are formed integrally with the second scan lines M.

The above term “end portions of the second scan lines M” includes not just the ends of the second scan lines M but also a range along each second scan line M from the end of the second scan line M to the TFT 2 that is connected closest to the end of the second scan line M. The term “end portions” does not indicate positions in the arrangement of the second scan lines M on the glass substrate 50.

Each of the signal lines D is connected to the signal processing section 35. The signal processing section 35 is equipped with an amplification circuit and a sample and hold circuit (neither of which is shown in the drawings) for each of the individual signal lines D. Each amplification circuit amplifies inputted electronic signals. After being amplified by the amplification circuits, the electronic signals transmitted through the individual signal lines D are retained at the sample and hold circuits. At the output side of the sample and hold circuits, a multiplexer and an analog-to-digital (A/D) converter (neither of which is shown in the drawings) are connected in this order. The electronic signals retained at the respective sample and hold circuits are sequentially (serially) inputted to the multiplexer, and are converted to digital image data by the A/D converter.

The image memory 36 stores image data outputted from the A/D converter of the signal processing section 35. The image memory 36 has a storage capacity capable of storing a predetermined number of frames of image data. Each time a radiation image is imaged, image data obtained by the imaging is sequentially stored in the image memory 36.

The control circuit 37 outputs control signals to the signal processing section 35 indicating timings of signal processing, and outputs controls signals to the scan line driving circuit 30 indicating timings of the output of driving signals. The control circuit 37 includes a microcomputer, and is provided with a central processing unit (CPU) and a memory including read-only memory (ROM) and random access memory (RAM), and a non-volatile storage section formed of flash memory or the like.

The radiographic imaging device 100 according to the present exemplary embodiment is provided with a radiation amount acquisition function that, in order to detect a radiation irradiation condition, acquires information representing a radiation amount of radiation irradiated from the radiation irradiation device 204. This radiation amount acquisition function is realized by, for example, sensors for radiation amount acquisition being provided in the radiation detector 20 and signals outputted from these sensors being read out and analyzed.

Herebelow, radiation image imaging operations by the radiographic imaging device 100 according to the present exemplary embodiment are described. The radiographic imaging device 100 starts an imaging operation of a radiation image when the above-mentioned radiation amount acquisition function detects the start of an irradiation of radiation from the radiation irradiation device 204. When the imaging operation is started, charges are accumulated in the sensors 61 of the pixels 60 of the radiation detector 20 in accordance with the irradiation of radiation thereon. The charges accumulated in the sensors 61 are outputted to the signal lines D via the TFTs 1 or the TFTs 2, and image data is generated at the signal processing section 35. The generated image data is stored in the image memory 36.

The radiographic imaging device 100 images the radiation image in either the high resolution mode or the low resolution mode in accordance with control signals provided from the system control device 202.

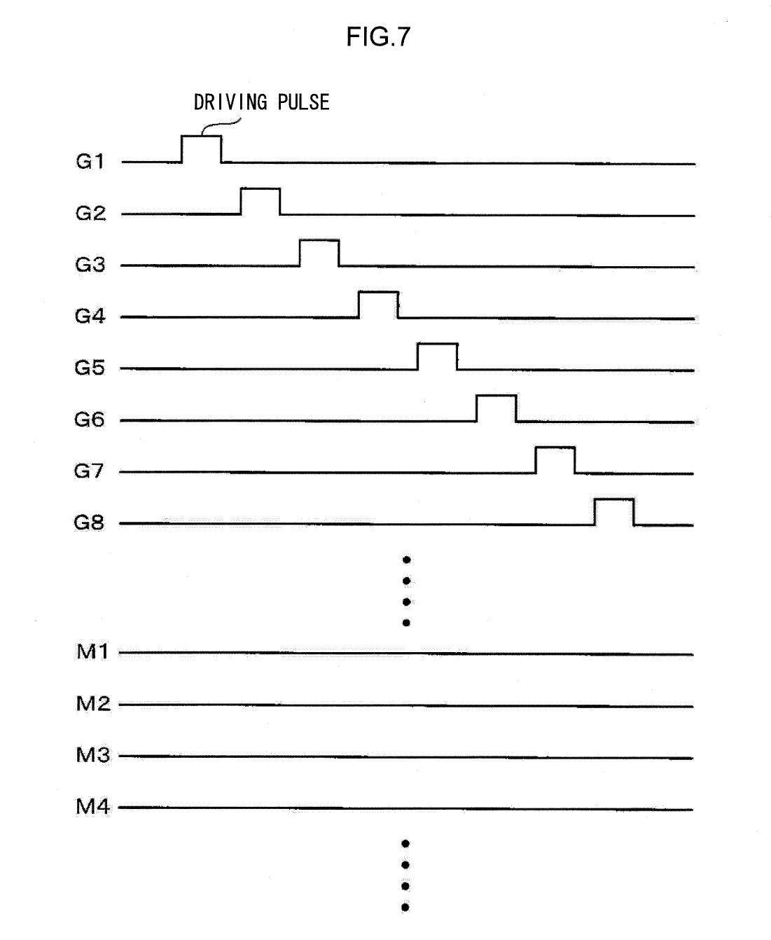

FIG. 7 is a timing chart of driving signals outputted from the scan line driving circuit 30 in a case in which the high resolution mode is selected.

In the high resolution mode, the first driving signal generation circuit 31 of the scan line driving circuit 30 provides driving pulses sequentially to the first scan lines G1, G2, G3, etc. When a driving pulse is provided to the first scan line G1, each of the TFTs 1 connected to the first scan line G1 turns ON, and the charges accumulated in the sensors 61 in pixels 60(1) to 60(4) are outputted to the signal lines D1 to D4, respectively. Then, when a driving pulse is provided to the first scan line G2, each of the TFTs 1 connected to the first scan line G2 turns ON, and the charges accumulated in the sensors 61 in pixels 60(5) to 60(8) are outputted to the signal lines D1 to D4, respectively. In this manner, in the high resolution mode the charges accumulated in the sensors 61 in the pixels 60 are outputted to mutually different signal lines D for the different pixels. Meanwhile, the second driving signal generation circuit 32 of the scan line driving circuit 30 does not generate driving signals in the high resolution mode. Therefore, all of the TFTs 2 connected to the respective second scan lines M stay in the OFF state in the high resolution mode.

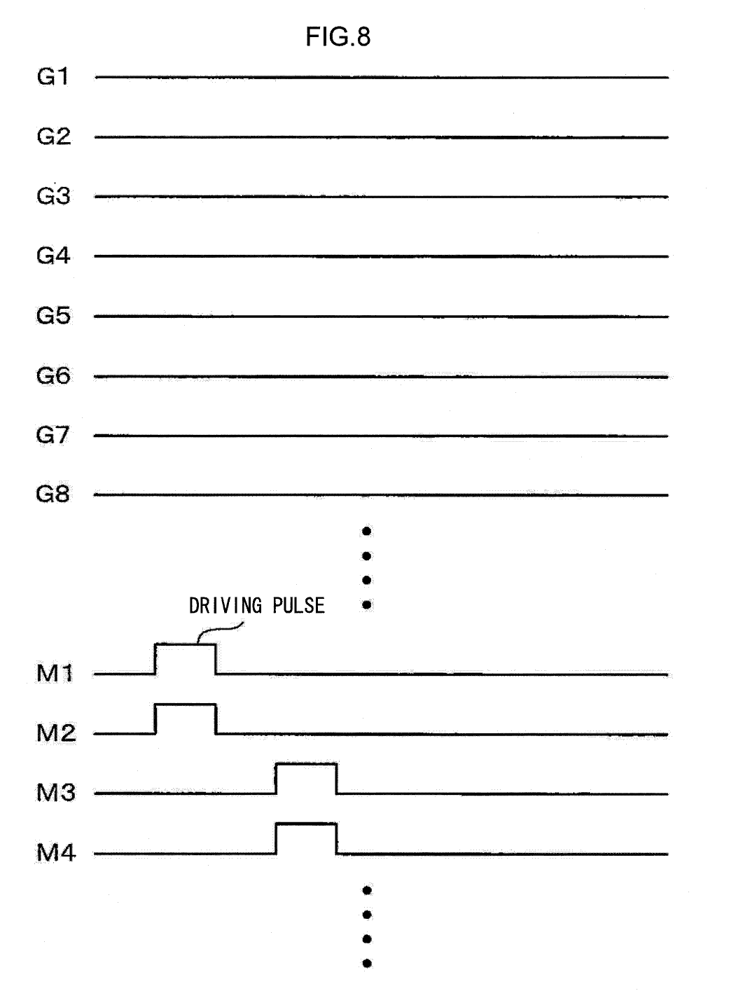

FIG. 8 is a timing chart of driving signals outputted from the scan line driving circuit 30 in a case in which the low resolution mode is selected.

In the low resolution mode, the second driving signal generation circuit 32 of the scan line driving circuit 30 provides driving pulses sequentially to the pair formed by the second scan lines M1 and M2, and the pair formed by the second scan lines M3 and M4, etc. That is, the same driving signals are provided at the same timings to the second scan lines M1 and M2, and then the same driving signals are provided at the same timings to the second scan lines M3 and M4. When a driving pulse is provided to the second scan lines M1 and M2, each of the TFTs 2 connected to the second scan lines M1 and M2 turns ON, and the charges accumulated in the sensors 61 in pixels 60(1) to 60(16) are outputted to the signal lines D1 to D5.

More specifically, for example, the charges accumulated in the sensors 61 of the four pixels 60(2), 60(3), 60(6) and 60(7) that are connected to the second scan line M1 and that are adjacent to one another in the direction in which the scan lines G and M extend and in the direction in which the signal lines D extend are respectively simultaneously outputted via the TFTs 2 in those pixels to the signal line D3. Further in this example, the charges accumulated in the sensors 61 of the four pixels 60(9), 60(10), 60(13) and 60(14) that are connected to the second scan line M2 and that are adjacent to one another in the direction in which the scan lines G and M extend and in the direction in which the signal lines D extend are respectively simultaneously outputted via the TFTs 2 in those pixels to the signal line D2.

Then, when a driving pulse is provided to the second scan lines M3 and M4, each of the TFTs 2 connected to the second scan lines M3 and M4 turns on, and the charges accumulated in the sensors 61 in pixels 60(17) to 60(32) are outputted to the signal lines D1 to D5. More specifically, for example, the charges accumulated in the sensors 61 of the four pixels 60(18), 60(19), 60(22) and 60(23) that are connected to the second scan line M3 and that are adjacent to one another in the direction in which the scan lines G and M extend and in the direction in which the signal lines D extend are respectively simultaneously outputted via the TFTs 2 in those pixels to the signal line D3. Further in this example, the charges accumulated in the sensors 61 of the four pixels 60(25), 60(26), 60(29) and 60(30) that are connected to the second scan line M4 and that are adjacent to one another in the direction in which the scan lines G and M extend and in the direction in which the signal lines D extend are respectively simultaneously outputted via the TFTs 2 in those pixels to the signal line D2.

Meanwhile, the first driving signal generation circuit 31 of the scan line driving circuit 30 does not provide driving signals to any of the first scan lines G in the low resolution mode. Therefore, all of the TFTs 1 connected to the respective first scan lines G stay in the OFF state in the low resolution mode.

In this manner, in the low resolution mode the charges accumulated in the sensors 61 of a set of four pixels that are connected to the same second scan line M and that are adjacent to one another in the direction in which the scan lines G and M extend and the direction in which the signal lines D extend are simultaneously outputted to the same signal line D. That is, in the low resolution mode, the composite pixels 70 are configured by combinations of four individual pixels of the high resolution mode. In other words, four pixels in the high resolution mode become a single pixel in the low resolution mode, and the resolution in the low resolution mode is a quarter of the resolution in the high resolution mode. Further, in the present exemplary embodiment, because a driving pulse is provided simultaneously to the pair of second scan lines M1 and M2 and charges are read simultaneously from the pixels 60 of four rows, the frame rate in the low resolution mode is four times that in the high resolution mode. Thus, a high frame rate is achieved.

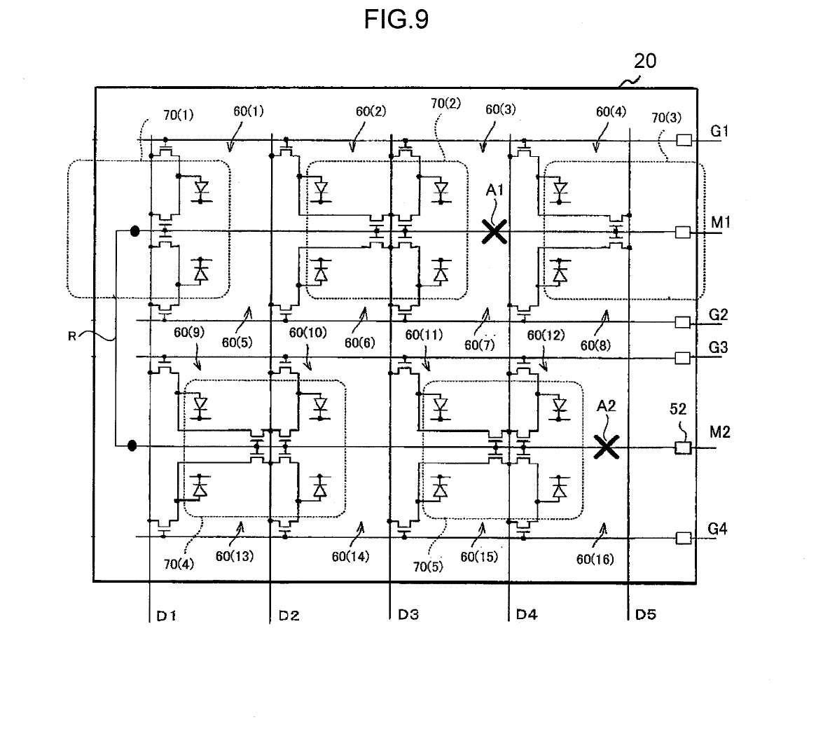

FIG. 9 is a partial structural diagram of the radiation detector 20 illustrating a case in which a breakage in a second scan line M has occurred. As an example, a case in which a breakage occurs at point A1 of the second scan line M1 between the composite pixels 70(2) and 70(3) is described below. In this case, a driving signal that the scan line driving circuit 30 outputs to the second scan line M1 is provided as far as the composite pixel 70(3), but is not provided to the composite pixels 70(1) and 70(2) at the far side of the composite pixel 70(3). However, the driving signal that the scan line driving circuit 30 outputs to the second scan line M2 is provided to the composite pixels 70(1) and 70(2) via the redundant line R. Therefore, even in the case in which a breakage has occurred at point A1, an occurrence of defective pixels may be avoided. If the redundant line R were not present, the composite pixels 70(1) and 70(2) would have become defective pixels.

As a further example, a case in which a breakage occurs at point A2 of the second scan line M2 between the composite pixel 70(5) and the connection terminal 52 thereof is described below. In this case, a driving signal that the scan line driving circuit 30 outputs to the second scan line M2 is not provided to any of the composite pixels 70 on the second scan line M2. However, the driving signal that the scan line driving circuit 30 outputs to the second scan line M1 is provided to each of the composite pixels 70 on the second scan line M2 via the redundant line R. Therefore, even in the case in which a breakage has occurred at point A2, an occurrence of defective pixels may be avoided. If the redundant line R were not present, all of the composite pixels 70 on the second scan line M2 would have become defective pixels.

Thus, according to the radiographic imaging device 100 of the present exemplary embodiment, even in a case in which a breakage has arisen on a second scan line M that is a transmission path for driving signals in the low resolution mode, driving signals that are outputted to the other second scan line M constituting the pair are provided via the redundant line R, and thus occurrences of defective pixels may be prevented. Moreover, because the redundant lines R are provided at the end portions at the opposite side of the second scan lines M from the ends that are connected to the scan line driving circuit 30, occurrences of defective pixels may be prevented regardless of breakage locations.

The scan line driving circuit 30 is a single configuration that includes the first driving signal generation circuit 31 that generates driving signals in the high resolution mode and the second driving signal generation circuit 32 that generates driving signals in the low resolution mode. Because the scan line driving circuit 30 is provided only at one side of the radiation detector 20, the device may be reduced in size. Therefore, the radiographic imaging device 100 according to the present exemplary embodiment may be excellently employed in a portable electronic cassette. Further, because the scan line driving circuit 30 is disposed only at one side of the radiation detector 20, structural portions that implement various additional functions may be provided at the opposite side of the radiation detector 20 from the side at which the scan line driving circuit 30 is disposed without leading to a reduction of the imaging area. Further, because the scan line driving circuit 30 is a single configuration capable of handling both the modes, the high resolution mode and the low resolution mode, an increase in a wiring burden due to routing of the first scan lines G and the second scan lines M on the radiation detector 20 may be prevented.

In the present exemplary embodiment, a case is illustrated in which each redundant line R is configured by a conductive body formed as a film on the glass substrate 50. However, a redundant line R may be configured to include a flexible cable, and may be configured to include a flexible substrate. When at least a portion of a redundant line R is configured by a flexible member, there is no need to reserve space for the redundant line R to extend along the glass substrate 50, and the device may be further reduced in size. For example, in a case in which another member (for example, a control circuit or the like) is disposed on the radiation detector, the redundant line R may be extended as far as the other member by the redundant line R featuring flexibility.

### Second Exemplary Embodiment

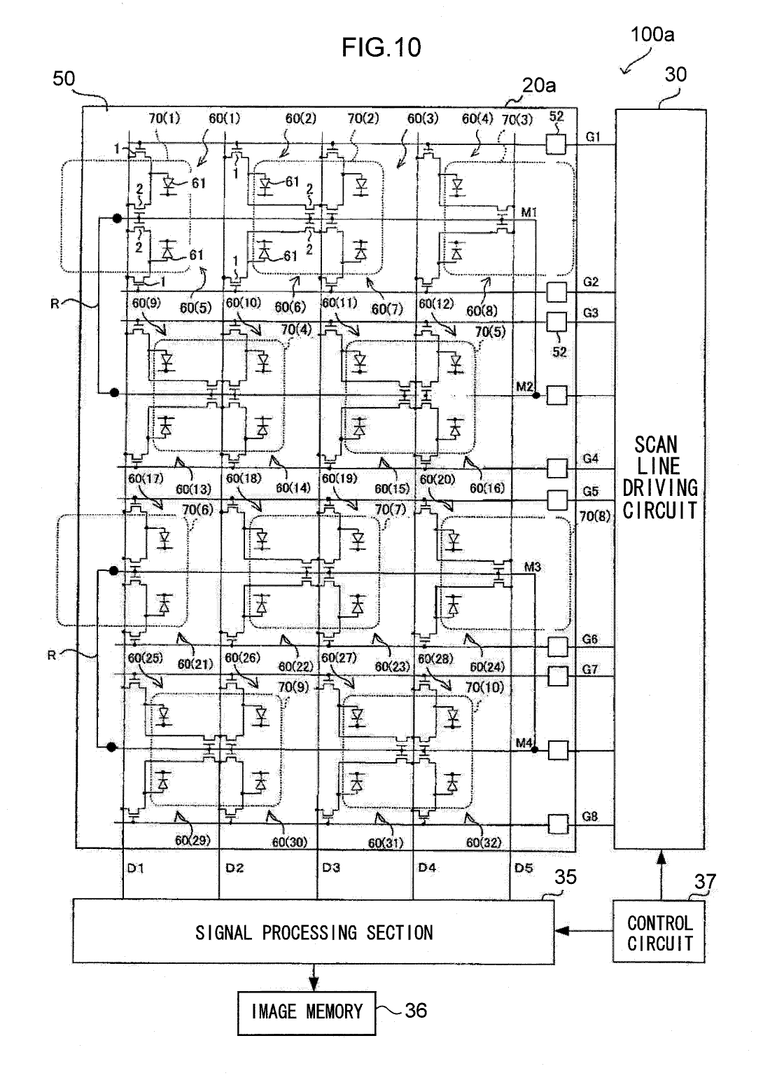

FIG. 10 is a structural diagram showing electronic configurations of a radiographic imaging device 100a in accordance with a second exemplary embodiment of the present invention. The radiographic imaging device 100a according to the present exemplary embodiment differs from the first exemplary embodiment described above in the configuration of the second scan lines M in a radiation detector 20a. That is, in the present exemplary embodiment, the pair formed by the second scan lines M1 and M2 and the pair formed by the second scan lines M3 and M4 are each electrically connected to one another on the glass substrate 50.

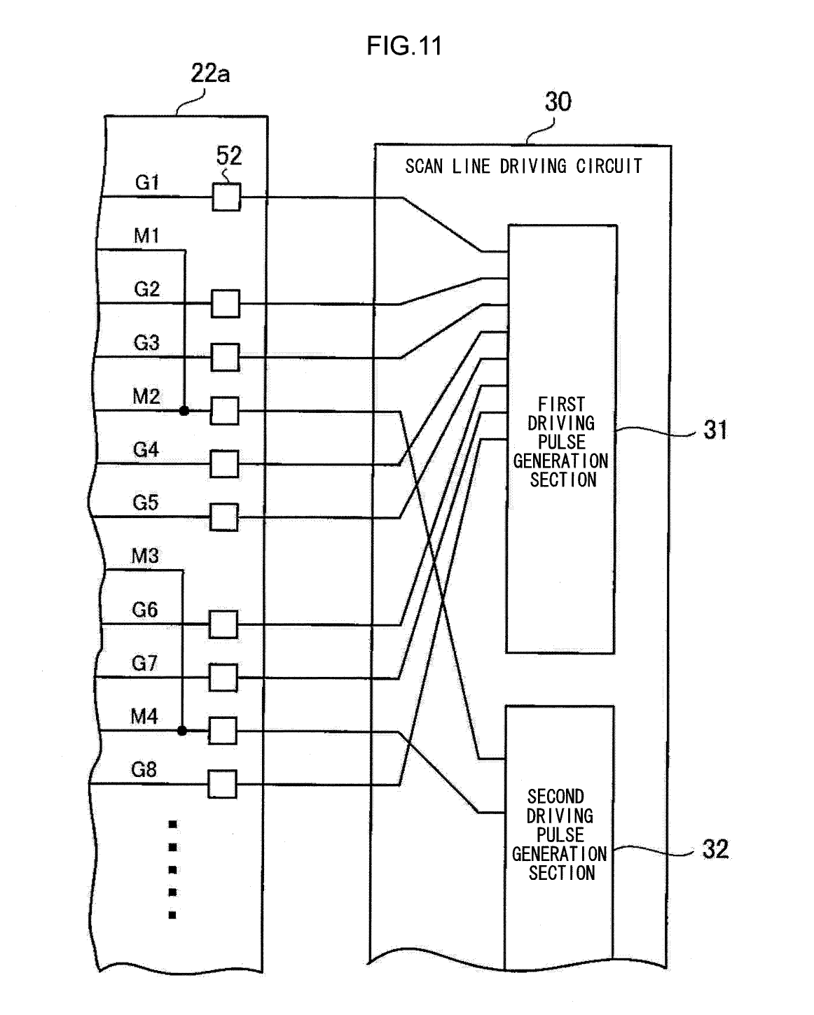

FIG. 11 is a diagram showing connection configurations between the radiation detector 20a and the scan line driving circuit 30 in accordance with the present exemplary embodiment. Each of the second scan lines M is connected to the scan line driving circuit 30 via a connection terminal 52 that is provided one for each of the pairs mentioned above. The scan line driving circuit 30 includes the first driving signal generation circuit 31 that operates when in the high resolution mode and the second driving signal generation circuit 32 that operates when in the low resolution mode. In the low resolution mode, the second driving signal generation circuit 32 outputs the same driving signals to the pairs of the second scan lines M.

As shown in FIG. 10, the second scan lines M1 and M2 and the second scan lines M3 and M4 that constitute pairs are each electrically connected to one another by the redundant lines R at the end portions that are at the opposite side from the end portions at the side at which the scan line driving circuit 30 is disposed.

Thus, in the radiographic imaging device 100a according to the present exemplary embodiment, the second scan lines M that constitute pairs are electrically connected, and in the low resolution mode the same driving signals are provided to the respective pairs. Therefore, the number of lines connecting the radiation detector 20a with the scan line driving circuit 30 may be half the number in the first exemplary embodiment. However, according to this configuration, capacitance loads at the scan line driving circuit 30 are increased, as a result of which rise times of the driving signals may be slower. In a case in which this would be a problem, a configuration that provides individual driving signals to the respective first scan lines M as in the first exemplary embodiment is preferable.

In the radiation detector 20a according to the present exemplary embodiment, similarly to the case of the first exemplary embodiment, imaging is possible in the high resolution mode and in the low resolution mode. Also similarly to the case of the first exemplary embodiment, even if a breakage occurs in a second scan line M, the driving signals outputted to the other second scan line M constituting that pair are provided via the redundant line R. Thus, occurrences of defective pixels may be prevented.

### Third Exemplary Embodiment

FIG. 12 is a structural diagram showing electronic configurations of a radiographic imaging device 100b in accordance with a third exemplary embodiment of the present invention. In the radiographic imaging device 100b according to the present exemplary embodiment, a first scan line driving circuit 30a is disposed adjacent to one of two opposing edges of a radiation detector 20b, and a second scan line driving circuit 30b is disposed adjacent to the other of the two edges. That is, the first scan line driving circuit 30a and the second scan line driving circuit 30b are disposed so as to sandwich the radiation detector 20b.

Each of the first scan lines G is connected to the first scan line driving circuit 30a via the respective connection terminal 52. The first scan line driving circuit 30a includes a driving signal generation circuit that generates driving signals when in the high resolution mode, and sequentially outputs driving pulses to each of the first scan lines G. The TFTs 1 turn ON in response to the driving pulses provided via the first scan lines G, and output charges accumulated in the sensors 61 to the signal lines D.

Each of the second scan lines M is connected to the second scan line driving circuit 30b via the respective connection terminal 52. The second scan line driving circuit 30b includes a driving signal generation circuit that generates driving signals when in the low resolution mode, and sequentially outputs driving pulses to each of the second scan lines M. The TFTs 2 turn ON in response to the driving pulses provided via the second scan lines M, and output charges accumulated in the sensors 61 to the signal lines D. Driving states in the high resolution mode and the low resolution mode are the same as in the first exemplary embodiment (see FIG. 7 and FIG. 8).

Thus, the present exemplary embodiment is configured in a mode in which the first scan line driving circuit 30a that operates when radiation images are being imaged in the high resolution mode and the second scan line driving circuit 30b that operates when radiation images are being imaged in the low resolution mode are separated from one another. In the present exemplary embodiment, the first scan line driving circuit 30a and the second scan line driving circuit 30b are disposed so as to sandwich the radiation detector 20b.

The pair constituted by the second scan lines M1 and M2, to which the same driving signals are provided at the same timings, are electrically connected by a redundant line R at the end portions at the side thereof at which the first scan line driving circuit 30a is disposed. Similarly, the pair constituted by the second scan lines M3 and M4 are electrically connected by a redundant line R at the end portions at the side thereof at which the first scan line driving circuit 30a is disposed.

Because the first scan lines G include wiring portions that extend toward the first scan line driving circuit 30a, the redundant lines R pass over the first scan lines G to be connected between the second scan lines M. Accordingly, in the present exemplary embodiment, the redundant lines R may be configured by jumper leads such as flexible cables or the like. Because the redundant lines R are configured by flexible cables or the like that are not formed integrally with the glass substrate 50, the same TFT substrate may be used in a case in which scan line driving circuits are disposed at two sides of the radiation detector as in the present exemplary embodiment and a case in which a scan line driving circuit is disposed only at one side of the radiation detector as in the first exemplary embodiment.

Thus, in the radiographic imaging device 100b of the present exemplary embodiment with this configuration too, similarly to the case of the first exemplary embodiment, imaging is possible in the high resolution mode and in the low resolution mode. Also similarly to the case of the first exemplary embodiment, even if a breakage occurs in a second scan line M, the driving signals outputted to the other second scan line M constituting that pair are provided via the redundant line R. Thus, occurrences of defective pixels may be prevented. In the present exemplary embodiment, because the scan line driving circuits are provided at both sides of the radiation detector 20b, the size of the device is larger than in the first exemplary embodiment. Therefore, the radiographic imaging device 100b according to the present exemplary embodiment is excellent for application to a radiographic imaging device of a built-in type, which is incorporated in a standing table for imaging a radiation image in a standing position and a reclining table for imaging a radiation image in a reclining position, or the like. Further, according to the radiographic imaging device 100b in accordance with the present exemplary embodiment, because the first scan line driving circuit 30a that operates when radiation images are being imaged in the high resolution mode and the second scan line driving circuit 30b that operates when radiation images are being imaged in the low resolution mode are configured as separate bodies, switching between the high resolution mode and the low resolution mode may be achieved in a shorter duration than in a case in which these scan line driving circuits are integrally configured.

In FIG. 12, the single first scan line driving circuit 30a is provided for all the first scan lines G, and the second single scan line driving circuit 30b is provided for all the second scan lines M. However, the scan line driving circuits may be provided one for each of predetermined numbers of the scan lines G and M. As an example, in the case in which 1024 of the first scan lines G are provided in the radiation detector 20b, the first scan line driving circuit 30a may be provided one for each 256 lines. In this case, four of the first scan line driving circuit 30a are provided. In the present exemplary embodiment, because the number of the second scan lines M is half the number of the first scan lines G, in the case in which the number of the first scan lines G is 1024, the number of the second scan lines M is 512. Therefore, in a case in which the second scan line driving circuit 30b is provided one for each 256 of the second scan lines M, two of the second scan line driving circuit 30b are provided. Thus, the number of the second scan line driving circuits 30b may be smaller than the number of the first scan line driving circuits 30a. Therefore, at a time of a reset operation that is conducted before the start of imaging of a radiation image in order to clear out charges accumulated in the sensors 61, power consumption may be reduced by using the second scan line driving circuits 30b that are fewer in number. Moreover, in a case in which the scan line driving circuits 30b are used to perform the reset operation, a duration required to complete the reset of the whole imaging area may be shorter than in a case in which the first scan line driving circuits 30a are used, and a duration from when an irradiation of radiation is started to when a charge accumulation mode is entered may be shortened.

### Fourth Exemplary Embodiment

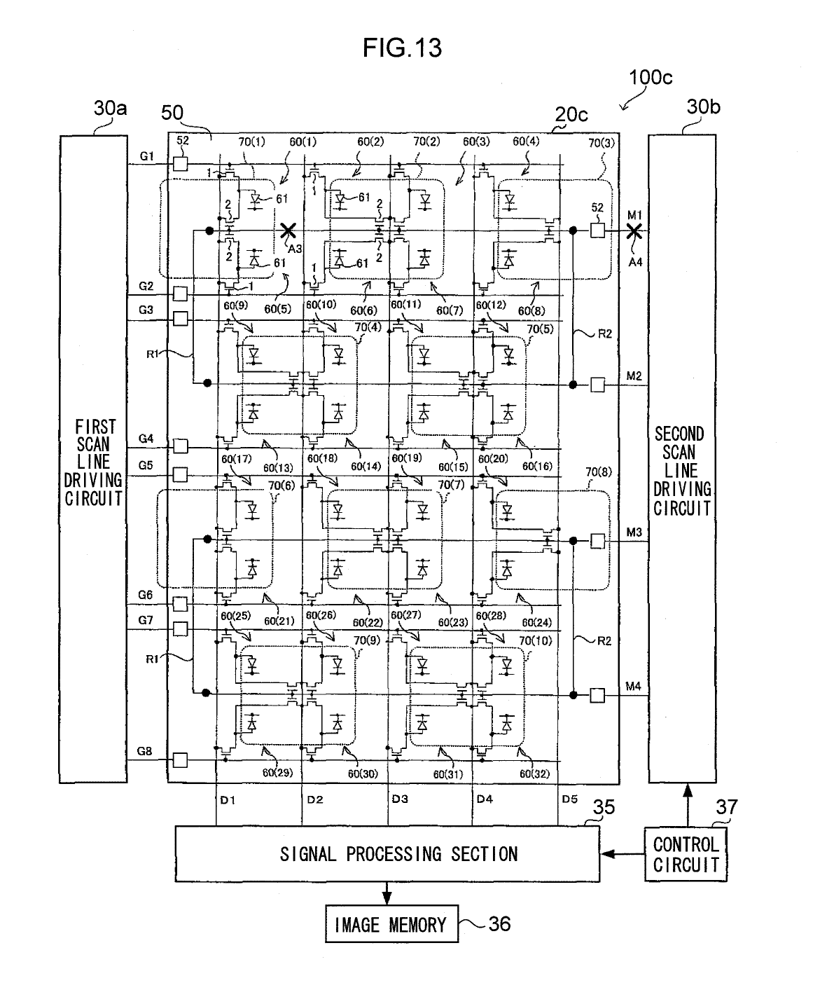

FIG. 13 is a structural diagram showing electronic configurations of a radiographic imaging device 100c in accordance with a fourth exemplary embodiment of the present invention. In the radiographic imaging device 100c according to the present exemplary embodiment, the first scan line driving circuit 30a is disposed adjacent to one of two opposing edges of a radiation detector 20c, and the second scan line driving circuit 30b is disposed adjacent to the other of the two edges. That is, the first scan line driving circuit 30a and the second scan line driving circuit 30b are disposed so as to sandwich the radiation detector 20c. The first scan line driving circuit 30a is connected to each of the first scan lines G at end portions at the opposite side thereof from connection ends at which the second scan lines M are connected to the second scan line driving circuit 30b. Meanwhile, the second scan line driving circuit 30b is connected to each of the second scan lines M at end portions at the opposite side thereof from connection ends at which the first scan lines G are connected to the first scan line driving circuit 30a. Driving states of the radiation detector 20c are the same as in the third exemplary embodiment.

The pair constituted by the second scan lines M1 and M2, to which the same driving signals are provided at the same timings, are electrically connected by a redundant line R1 at the end portions at the side thereof at which the first scan line driving circuit 30a is disposed. In the present exemplary embodiment, the second scan lines M1 and M2 are also connected by a redundant line R2 at the end portions at the side thereof at which the second scan line driving circuit 30b is disposed. Similarly, the pair constituted by the second scan lines M3 and M4 are electrically connected by a redundant line R1 at the end portions at the side thereof at which the first scan line driving circuit 30a is disposed, and are electrically connected by a redundant line R2 at the end portions at the side thereof at which the second scan line driving circuit 30b is disposed.

Thus, in the radiographic imaging device 100c according to the present exemplary embodiment, the respective scan lines M constituting pairs are electrically connected by the redundant lines R1 and R2 at both end portions thereof. Because the redundant lines are provided at plural locations, an occurrence of defective pixels may be prevented even if breakages occur at plural locations on a second scan line M. For example, as illustrated in FIG. 13, a case is described below in which a breakage occurs at point A3 on the second scan line M1, between the composite pixels 70(1) and 70(2), and a breakage occurs at point A4, between the second scan line driving circuit 30b and the connection terminal 52. In this case, the driving signals outputted by the second scan line driving circuit 30b to the second scan line M1 are not provided to any of the composite pixels 70 on the second scan line M1. However, the driving signals outputted to the second scan line M2 by the second scan line driving circuit 30b are provided via the redundant line R1 to the composite pixel 70(1) on the second scan line M1, and are provided via the redundant line R2 to the composite pixels 70(2) and 70(3) on the second scan line M1. Therefore, an occurrence of defective pixels may be prevented even in the case in which breakages occur at point A3 and point A4. If the redundant lines R1 and R2 were not present, all of the composite pixels 70 would have become defective pixels.

The present exemplary embodiment has a configuration in which the redundant lines R1 and R2 are provided at both end portions of the second scan lines M constituting a pair. However, the redundant line R2 may be provided at a middle portion of the second scan lines M, between the composite pixels. Furthermore, the redundant lines may be both disposed at the two end portions of the second scan lines M and disposed at middle portions between the composite pixels. That is, the redundant lines may be provided at three or more locations on the second scan lines M constituting the pair. Thus, even in a case in which breakages occur at plural points on a second scan line, an occurrence of defective pixels may be prevented or an occurrence of defective pixels may be reduced in scale because the number of redundant lines is increased. This configuration in which two or more redundant lines connect the second scan lines M constituting a pair may also be applied to the radiation detectors according to the first and second exemplary embodiments.

### Fifth Exemplary Embodiment

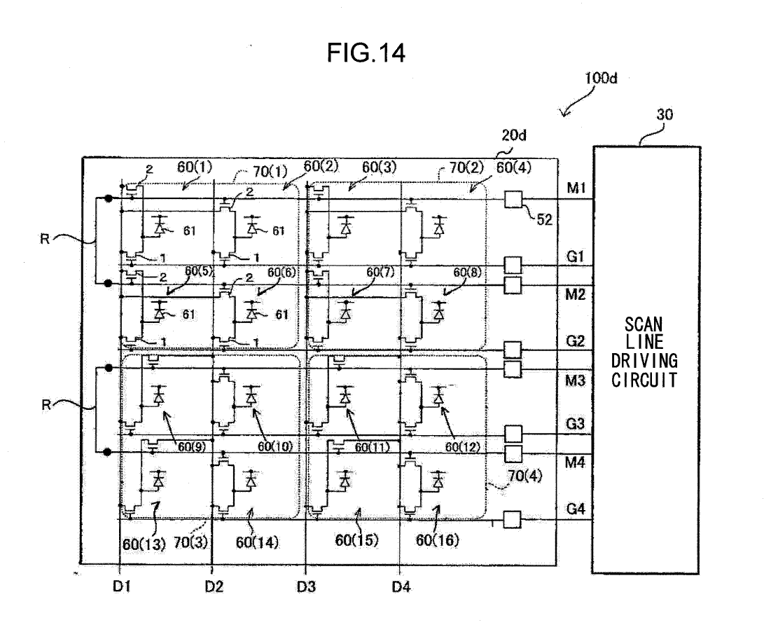

FIG. 14 is a structural diagram showing electronic configurations of a radiographic imaging device 100d in accordance with a fifth exemplary embodiment of the present invention. In a radiation detector 20d structuring the radiographic imaging device 100d according to the present exemplary embodiment, the mode of connection of the TFTs 2 that operate when in the low resolution mode to the second scan lines M and the signal lines D differs from the first to fourth exemplary embodiments described above.

The control terminals (gates) of the plural TFTs 1 that are driven when a radiation image is being imaged in the high resolution mode are connected to the respective first scan lines G. More specifically, the control terminals (gates) of the TFTs 1 in a plural number of the pixels 60 that are in a line along the direction in which the first scan lines G extend are connected to the same first scan line G. For example, in the example shown in FIG. 14, the control terminals (gates) of the TFTs 1 structuring pixels 60(1) to 60(4) are connected to the first scan line G1, and the control terminals (gates) of the TFTs 1 structuring pixels 60(5) to 60(8) are connected to the first scan line G2.

The control terminals (gates) of a plural number of the TFTs 2 that are driven when a radiation image is being imaged in the low resolution mode are connected to each of the second scan lines M. More specifically, the TFTs 2 in a plural number of the pixels 60 that are in a line along the direction in which the second scan lines M extend are connected to the same second scan line M. For example, in the example shown in FIG. 14, the gates of the TFTs 2 structuring pixels 60(1) to 60(4) are connected to the second scan line M1, and the control terminals (gates) of the TFTs 2 structuring pixels 60(5) to 60(8) are connected to the second scan line M2.

The output terminals of the TFTs 1 in a plural number of the pixels 60 that are in a line along the direction in which the signal lines D extend are connected to the same signal line D. For example, in the example shown in FIG. 14, the output terminals of the TFTs 1 in pixels 60(1), 60(5), 60(9) and 60(13) are connected to the signal line D1, and the output terminals of the TFTs 1 in pixels 60(2), 60(6), 60(10) and 60(14) are connected to the signal line D2.

The output terminals of the TFTs 2 in four pixels that are adjacent to one another in the direction in which the scan lines G and M extend and in the direction in which the signal lines D extend are connected to the same signal line D. For example, in the example shown in FIG. 14, the output terminals of the TFTs 2 that constitute the composite pixel 70(1) formed of the pixels 60(1), 60(2), 60(5) and 60(6) are connected to the signal line D1, the output terminals of the TFTs 2 that constitute the composite pixel 70(3) formed of the pixels 60(9), 60(10), 60(13) and 60(14) are connected to the signal line D2, the output terminals of the TFTs 2 that constitute the composite pixel 70(2) formed of the pixels 60(3), 60(4), 60(7) and 60(8) are connected to the signal line D3, and the output terminals of the TFTs 2 that constitute the composite pixel 70(4) formed of the pixels 60(11), 60(12), 60(15) and 60(16) are connected to the signal line D4.

In the low resolution mode, the scan line driving circuit 30 provides driving pulses sequentially to the pair formed of the second scan lines M1 and M2 and to the pair formed of the second scan lines M3 and M4. That is, the same driving signals are provided at the same timings to the second scan lines M1 and M2, and then the same driving signals are provided at the same timings to the second scan lines M3 and M4.

When a driving pulse is provided to the second scan lines M1 and M2, each of the TFTs 2 connected to the second scan lines M1 and M2 turns ON, and the charges accumulated in the sensors 61 in pixels 60(1) to 60(8) are outputted to the signal lines D1 and D3. More specifically, for example, the charges accumulated in the sensors 61 of the four pixels 60(1), 60(2), 60(5) and 60(6) that are adjacent to one another in the direction in which the scan lines G and M extend and the direction in which the signal lines D extend are respectively outputted via the TFTs 2 in those pixels to the signal line D1. Further in this example, the charges accumulated in the sensors 61 of the four pixels 60(3), 60(4), 60(7) and 60(8) that are adjacent to one another in the direction in which the scan lines G and M extend and the direction in which the signal lines D extend are respectively outputted via the TFTs 2 in those pixels to the signal line D3.

Then, when a driving pulse is provided to the second scan lines M3 and M4, each of the TFTs 2 connected to the second scan lines M3 and M4 turns ON, and the charges accumulated in the sensors 61 in pixels 60(9) to 60(16) are outputted to the signal lines D2 and D4. More specifically, for example, the charges accumulated in the sensors 61 of the four pixels 60(9), 60(10), 60(13) and 60(14) that are adjacent to one another in the direction in which the scan lines G and M extend and the direction in which the signal lines D extend are respectively outputted via the TFTs 2 in those pixels to the signal line D2. Further in this example, the charges accumulated in the sensors 61 of the four pixels 60(11), 60(12), 60(15) and 60(16) that are adjacent to one another in the direction in which the scan lines G and M extend and the direction in which the signal lines D extend are respectively outputted via the TFTs 2 in those pixels to the signal line D4.

Operations when in the high resolution mode are the same as in the case of the first exemplary embodiment, so are not described here.

Thus, in the radiographic imaging device 100d according to the present exemplary embodiment, in the low resolution mode the charges accumulated in the sensors 61 of a set of four pixels that are adjacent to one another in the direction in which the scan lines G and M extend and the direction in which the signal lines D extend are simultaneously outputted to the same signal line D. That is, in the low resolution mode, the composite pixels 70 are configured by combinations of four individual pixels of the high resolution mode. In other words, four pixels in the high resolution mode become a single pixel in the low resolution mode, and the resolution in the low resolution mode is a quarter of the resolution in the high resolution mode. Further, in the present exemplary embodiment, because a driving pulse is provided simultaneously to the pair of second scan lines M1 and M2 and charges are read simultaneously from the pixels 60 of two rows, the frame rate in the low resolution mode is two times that in the high resolution mode. Thus, a high frame rate is achieved.

The second scan lines M1 and M2 that constitute a pair to which the same driving signals are provided from the scan line driving circuit 30 simultaneously are electrically connected to one another by a redundant line R, at the end portions of the scan lines M1 and M2 that are at the opposite side thereof from the end portions at the side at which the scan line driving circuit 30 is disposed. Similarly, the pair formed of the second scan lines M3 and M4 are electrically connected to one another by a redundant line R at the end portions of the scan lines M3 and M4 that are at the opposite side thereof from the end portions at the side at which the scan line driving circuit 30 is disposed. Thus, the same as in the exemplary embodiments described above, even in a case in which a breakage has occurred on a second scan line M that is a transmission path for driving signals in the low resolution mode, driving signals that are outputted to the other second scan line M constituting the pair are provided via the redundant line R, and thus occurrences of defective pixels may be prevented.

The exemplary embodiments described above illustrate a radiographic imaging device of an indirect conversion type in which irradiated radiation is converted to light by a scintillator to image a radiation image. However, the present invention is also applicable to a radiographic imaging device of a direct conversion type that directly converts radiation to charges in a semiconductor layer of amorphous selenium or the like.

Further, the exemplary embodiments described above illustrate a case in which four pixels in the high resolution mode serve as a single pixel in the low resolution mode. However, the resolution in the low resolution mode may be altered as appropriate, by modifying connection configurations between the TFTs 2 and the second scan lines M and signal lines D to increase the number of sensors from which charges are simultaneously read out into the same signal line (in other words, the number of the pixels 60 that constitute each composite pixel 70). In this case, if the number of the second scan lines to which the same or identical driving signals are provided in the low resolution mode is three or more, it is appropriate to provide redundant lines so as to connect each of these second scan lines to one another.

Configurations of the respective exemplary embodiments described above may be combined as appropriate.

In the exemplary embodiments described above, a case is illustrated in which X-rays are detected as the radiation that is the object of detection. However, the present invention is not limited thus. For example, the radiation that is the object of detection may be any of visible light, ultraviolet rays, infrared rays, alpha rays, gamma rays and the like.

In addition, configurations of the radiographic imaging system, configurations of the radiographic imaging device and so forth described in the above exemplary embodiments are examples, and may be suitably modified within a technical scope not departing from the spirit of the present invention.

The disclosures of Japanese Patent Application No. 2012-123627 are incorporated into the present specification by reference in their entirety.

All references, patent applications and technical specifications cited in the present specification are incorporated by reference into the present specification to the same extent as if the individual references, patent applications and technical specifications were specifically and individually recited as being incorporated by reference.

What is claimed is:

1. A radiographic imaging device comprising:
a plurality of first scan lines and a plurality of second scan lines extending in a first direction;
a plurality of signal lines extending in a second direction that crosses the first direction;
a plurality of first switching elements provided in correspondence with intersection portions between the plurality of signal lines and the plurality of first scan lines, control terminals of the first switching elements being connected to the corresponding first scan lines and output terminals of the first switching elements being connected to the corresponding signal lines;
a plurality of sensors, each of which is connected to an input terminal of a respective one of the first switching elements and generates charges in accordance with intensities of irradiated radiation or intensities of light corresponding to the radiation;
a plurality of second switching elements, each of which includes an input terminal connected to a respective one of the sensors and a control terminal connected to one of the second scan lines, respective output terminals of the plurality of the second switching elements, whose respective input terminals are connected to a plurality of the sensors, which sensors are adjacent in the first direction and the second direction, being connected to the same signal line;
a first driving signal provision section that provides driving signals to the plurality of first scan lines;
a second driving signal provision section that provides driving signals to the plurality of second scan lines; and
a connection portion that electrically connects to one another a plurality of the second scan lines to which the same or identical driving signals are provided by the second driving signal provision section.

2. The radiographic imaging device according to claim 1, wherein the connection portion is provided at second end portions of the second scan lines, the second end portions being at an opposite side of the second scan lines from first end portions at a side thereof at which the second driving signal provision section is connected.

3. The radiographic imaging device according to claim 2, wherein the connection portion is provided at the first end portions and the second end portions of the second scan lines.

4. The radiographic imaging device according to claim 2, wherein the connection portion is provided between the first end portions and the second end portions of the second scan lines.

5. The radiographic imaging device according to claim 1, wherein the connection portion is formed integrally with each of the second scan lines.

6. The radiographic imaging device according to claim 1, wherein the connection portion includes at least a portion of a flexible cable and a flexible substrate.

7. The radiographic imaging device according to claim 1, wherein:
the first driving signal provision section provides driving signals to each of the first scan lines when in a first imaging mode, and
the second driving signal provision section provides driving signals to each of the second scan lines when in a second imaging mode.

8. The radiographic imaging device according to claim 1, wherein the first driving signal provision section and the second driving signal provision section are formed in a single package.

9. The radiographic imaging device according to claim 8, wherein the first driving signal provision section and the second driving signal provision section are connected to end portions of the plurality of first scan lines and the plurality of second scan lines.

10. The radiographic imaging device according of claims 1 to 7, wherein the first driving signal provision section and the second driving signal provision section are separately provided.

11. The radiographic imaging device according to claim 10, wherein the first driving signal provision section is connected to each of the plurality of first scan lines at end portions at the opposite side thereof from connection portions that connect the plurality of second scan lines with the second driving signal provision section.

12. The radiographic imaging device according to claim 1, further comprising a signal processing section that is connected to each of the plurality of signal lines and that generates a radiation image in accordance with charges read out to the signal lines from the plurality of sensors in response to driving that turns ON the first switching elements or the second switching elements.

13. A radiation detector comprising:
a plurality of first scan lines and a plurality of second scan lines extending in a first direction;
a plurality of signal lines extending in a second direction that crosses the first direction;
a plurality of first switching elements provided in correspondence with intersection portions between the plurality of signal lines and the plurality of first scan lines, control terminals of the first switching elements being connected to the corresponding first scan lines and output terminals of the first switching elements being connected to the corresponding signal lines;
a plurality of sensors, each of which is connected to an input terminal of a respective one of the first switching elements and that generates charges in accordance with intensities of irradiated radiation or intensities of light corresponding to the radiation;
a plurality of second switching elements, each of which includes an input terminal connected to a respective one of the sensors and a control terminal connected to one of the second scan lines, respective output terminals of the plurality of the second switching elements, whose respective input terminals are connected to a plurality of the sensors, which sensors are adjacent in the first direction and the second direction, being connected to the same signal line; and
a connection portion that electrically connects to one another a plurality of the second scan lines to which the same or identical driving signals are provided.
